(19) United States
(12) Patent Application Publication
(10) Pub. No.: US 2025/0049651 A1
(43) Pub. Date: Feb. 13, 2025

(54) TWO-PHASE MAKE-UP REMOVER

(71) Applicant: BEIERSDORF AG, Hamburg (DE)

(72) Inventor: Heba SACCA, Hamburg (DE)

(21) Appl. No.: 18/719,128

(22) PCT Filed: Nov. 8, 2022
(86) PCT No.: PCT/EP2022/081086

§ 371 Date: Jun. 12, 2024

(30) Foreign Application Priority Data
Dec. 17, 2021 (DE) 20 2021 003 812.4

(51) Int. Cl. A61K 8/03; A61K 8/20; A61K 8/31; A61K 8/37; A61K 8/60; A61K 8/92; A61Q 1/14

(52) U.S. Cl. CPC A61K 8/03; A61K 8/20; A61K 8/31; A61K 8/37; A61K 8/604; A61K 8/92; A61Q 1/14; A61K 2800/34

(57) ABSTRACT

The present invention belongs to the cosmetic field and relates to a two-phase make-up remover.

The present invention belongs to the field of cosmetics and relates to a two-phase makeup remover.

A beautiful and attractive appearance is desired by many people. A typical sign of such an appearance is healthy and smooth-looking skin. In order to make the skin look beautiful or to maintain its appearance, many people's daily routine includes the application to the skin of decorative cosmetic products such as mascara or foundations. However, before going to bed most people remove the decorative cosmetic products again. The products are conventionally removed using a two-phase makeup remover which has an aqueous phase and an oil phase in the form of two clearly separate phases.

The two-phase makeup remover is applied by shaking the product in order to mix both phases and then transferring some of the mixture to a suitable cloth or pad. The cloth or pad is then used to release the makeup from the skin and absorb it.

Various two-phase makeup removers are known from the prior art. Typical examples are described in U.S. Pat. No. 5,165,917A.

A typical ingredient of these products is silicone oils or silicone compounds. These compounds enable effective cleansing and rapid and effective phase separation after mixing the ingredients. This means that no “sweating” can be observed and the phases do not become cloudy. The separation time, that is to say the time until a phase boundary becomes visible, is generally up to 60 minutes. The clouding of the phases should disappear within 12 to 24 hours; the sooner the better.

For the purposes of the present disclosure, the term “sweating” is understood to mean oil droplets adhering to the surface of the bottle in the water phase section.

For the purposes of the present disclosure, the term “cloudy” is understood to mean not clear or transparent to the human eye.

For the purposes of the present disclosure, the term “silicone oils and silicone compounds” is understood to mean ingredients containing a dimethylsiloxane unit.

However, the use of silicone oils and silicone compounds has been controversial in recent years, meaning that many consumers prefer to use products that do not contain these compounds.

The lack of silicone oils and silicone compounds impairs the properties of the two-phase makeup remover. In terms of the separation properties, the separation time after mixing is often prolonged. Furthermore, sweating effects are frequently observed. Moreover, the separate phases become increasingly cloudy, which makes it impossible to maintain a clean and attractive appearance during the use cycle of the product. In addition, it is found that the ability to remove waterproof makeup is generally reduced. In particular, it is desirable to avoid clouding of the formulations in both phases 24 hours after shaking/mixing the phases.

It was accordingly an object of the present invention to address, solve and/or reduce the defects of makeup removers that do not contain any silicone oils or silicone compounds.

It has surprisingly been established by the application that the present invention can achieve these objects.

A first subject of the present invention is a two-phase cosmetic cleansing composition which is not an emulsion, comprising:
a) an aqueous phase and
b) an oil phase,
wherein the volume ratio between the aqueous phase and the oil phase is in the range from 3:1 to 1:3, the oil phase being characterized in that
it has an interfacial tension with water in the range from 30 to 39 mN/m,
a density in the range from 0.76 to 0.81 g/cm³,
the oil phase is free of silicone oils and silicone compounds.

It has surprisingly been found that 24 h after shaking the composition the phase boundary is discretely visible and the phases of the separated composition display no clouding and no sweating effects.

Accordingly, the objects of the present invention are achieved.

Unless stated otherwise, all percentages by weight (% by weight) reported below are based on the total weight of the cosmetic cleansing composition. Where ratios of certain components are disclosed in the following description, these ratios relate to ratios by weight of the components unless stated otherwise.

Unless stated otherwise, all figures and measurements have been conducted under “standard conditions”. The term “standard conditions” refers to 20° C., 1013 hPa and a relative humidity of 50%.

In the following description, the terms “according to the invention”, “preferred according to the invention”, and so on always relate to the cosmetic composition according to the invention, the use according to the invention and the process according to the invention.

The term “free of” for the purposes of the present disclosure means that the proportion of the respective substance is less than 0.05% by weight, preferably less than 0.01% by weight and very particularly preferably 0% by weight, based on the total weight, of the respective phase of the composition. Accordingly, the weight percentage values for silicone oil and silicone compounds relate to the total weight of the oil phase. This definition ensures that entrainment or contamination with these substances is not designated as “free of” according to the invention.

The term “skin” refers exclusively to human skin.

Emulsifiers are understood to be all substances which are listed in the International Cosmetic Ingredient Dictionary and Handbook, Thirteenth Edition 2010, (ISBN 1-882621-47-6) under the designation “emulsifier”. Surfactants are understood to be all substances which are listed in the International Cosmetic Ingredient Dictionary and Handbook, Thirteenth Edition 2010, (ISBN 1-882621-47-6) under the designation “surfactant”.

According to the invention, it is preferable when the volume ratio between the aqueous phase and the oil phase is 2:1 to 1:2, particularly preferably 1.5:1 to 1:1.5 and very particularly preferably 1.3:1 to 1:1.3.

It is further preferable when the aqueous phase contains water in a total amount of at least 60% by weight, more preferably at least 65% by weight, more preferably at least 70% by weight, more preferably at least 75% by weight, more preferably at least 80% by weight, particularly preferably at least 85% by weight and very particularly preferably at least 90% by weight, calculated on the basis of the total weight of the aqueous phase.

It is further preferable when the aqueous phase contains water in a total amount of less than 99% by weight, particularly preferably less than 98% by weight, particularly preferably less than 97% by weight, particularly preferably less than 96% by weight and most preferably less than 95.5% by weight, calculated on the basis of the total weight of the aqueous phase.

Preferably, the aqueous phase contains at least one surfactant. The surfactant may be selected from anionic, nonionic, amphoteric and cationic surfactants, with nonionic and amphoteric surfactants being preferred.

It is preferable here when the total amount of the surfactants is in the range from 0.01% to 2% by weight, particularly preferably from 0.02% to 1% by weight, particularly preferably from 0.025% to 0.5% by weight and very particularly preferably from 0.03% to 0.4% by weight, calculated on the basis of the total weight of the aqueous phase.

Preferred anionic surfactants according to the invention include:
linear and branched fatty acids having 8 to 30 carbon atoms (soaps),
ether carboxylic acids of formula R—O—(CH₂—CH₂O)ₓ—CH₂—COOH, in which R is a linear or branched, saturated or unsaturated alkyl group having 8 to 30 carbon atoms and x=0 or ito 16,
acyl sarcosides having 8 to 24 carbon atoms in the acyl group,
acyl taurides having 8 to 24 carbon atoms in the acyl group,
acyl isethionates having 8 to 24 carbon atoms in the acyl group,
sulfosuccinic acid mono- and/or dialkyl esters having 8 to 24 carbon atoms in the alkyl group and sulfosuccinic acid monoalkyl polyoxyethyl esters having 8 to 24 carbon atoms in the alkyl group and 1 to 6 oxyethyl groups,
alpha-olefinsulfonates having 8 to 24 carbon atoms,
alkyl sulfate and/or alkyl polyglycol ether sulfate salts of formula R—(OCH₂—CH₂)ₓ—OSO₃-M+, in which R is a preferably linear or branched, saturated or unsaturated alkyl group having 8 to 30 carbon atoms, x=0 or 1 to 12, and M is an alkali metal ion or ammonium ion,
sulfonates of unsaturated fatty acids having 8 to 24 carbon atoms and 1 to 6 double bonds,
esters of tartaric acid and citric acid with alcohols which are adducts of about 2-15 molecules of ethylene oxide and/or propylene oxide onto fatty alcohols having 8 to 22 carbon atoms,
alkyl and/or alkenyl ether phosphates of the formula

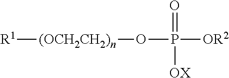

in which R¹ is preferably an aliphatic hydrocarbon group having 8 to 30 carbon atoms, R² is hydrogen, a (CH₂CH₂O)ₙR¹ group or X, n is numbers from 0 to 10 and X is hydrogen, an alkali metal or alkaline earth metal, or NR³R⁴R⁵R⁶, where R³ to R⁶ are independently a C₁ to C₄ hydrocarbon group.

Preferred anionic surfactants are ether carboxylic acids of the abovementioned formula, acyl sarcosides having 8 to 24 carbon atoms in the acyl group, sulfosuccinic acid mono- and/or dialkyl esters having 8 to 24 carbon atoms in the alkyl group and sulfosuccinic acid monoalkyl polyoxyethyl esters having 8 to 24 carbon atoms in the alkyl group and 1 to 6 oxyethyl groups, alpha-olefinsulfonates having 8 to 24 carbon atoms and/or alkyl sulfate salts and/or alkyl polyglycol ether sulfate salts of the abovementioned formula.

Particularly preferred anionic surfactants are straight-chain or branched alkyl ether sulfates containing an alkyl group having 8 to 18, in particular having 10 to 16, carbon atoms and 1 to 6, in particular 2 to 4, ethylene oxide units. Further particularly preferred anionic surfactants are straight-chain or branched alkylsulfonates comprising an alkyl group having 8 to 18 and in particular 10 to 16 carbon atoms. Particular preference is given to the sodium, magnesium and/or triethanolamine salts of linear or branched lauryl, tridecyl and/or myristyl sulfates having a degree of ethoxylation of 2 to 4.

Preferred amphoteric surfactants may be selected from compounds of the following formulae (i) to (v), in which the R group is in each case a straight-chain or branched, saturated or mono—or polyunsaturated alkyl or alkenyl group having 8 to 24 carbon atoms:

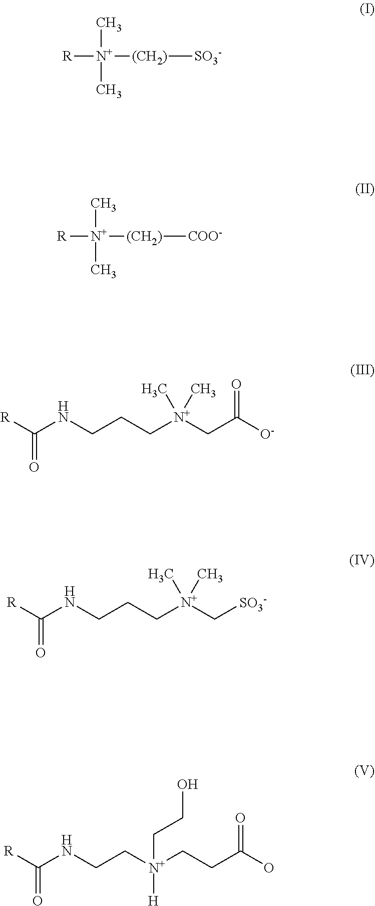

Particularly suitable amphoteric surfactants are alkylamidoalkyl betaines and/or alkyl ampho(di)acetates of the abovementioned formulae (I) to (V). Particularly suitable amphoteric surfactants are the surfactants known under the INCI names Sodium Cocoamphoacetate, Cocamidopropyl Betaine and Disodium Cocoamphodiacetate. Most preferred is Sodium Cocoamphoacetate.

It is further preferable when at least one nonionic surfactant is present in the aqueous phase. Preferred nonionic surfactants are selected from alkyl glucosides, preferably coco glucoside, caprylyl/capryl glucoside, decyl glucoside and lauryl glucoside. Yet further preference is given to coco glucoside and/or caprylyl/capryl glucoside. Caprylyl/capryl glucoside is most preferred.

Among all surfactants, it is most preferable when at least one nonionic surfactant, in particular an alkyl glucoside, is present, preferably coco glucoside, caprylyl/capryl glucoside, decyl glucoside and lauryl glucoside. Caprylyl/capryl glucoside is still most preferred.

It is preferable when the total amount of the nonionic surfactants is in the range from 0.02% to 1% by weight, particularly preferably from 0.02% to 0.5% by weight, particularly preferably from 0.1% to 0.4% by weight and very particularly preferably from 0.15% to 0.19% by weight, calculated on the basis of the total weight of the aqueous phase.

It is particularly preferable when caprylyl/capryl glucoside is present in the aqueous phase in a total amount in the range from 0.02% to 1% by weight, particularly preferably from 0.02% to 0.5% by weight, particularly preferably from 0.1% to 0.4% by weight and very particularly preferably 0.15% to 0.19% by weight, calculated on the basis of the total weight of the aqueous phase.

According to the invention, it is further preferable when the aqueous phase contains at least one complexing agent, which is preferably EDTA.

It is further preferable when the aqueous phase of the composition contains at least one preservative. Preferred preservatives are selected from the group consisting of phenoxyethanol, ethylhexylglycerin and benzethonium chloride. Particular preference is given to phenoxyethanol and/or benzethonium chloride. Benzethonium chloride is most preferred.

If phenoxyethanol is present, it is preferable when the total amount of phenoxyethanol is in the range from 0.1% to 1.5% by weight, particularly preferably 0.2% to 1.2% by weight and very particularly preferably 0.4% to 0.8% by weight, calculated on the basis of the total weight of the aqueous phase.

If benzethonium chloride is present, it is preferable when the total amount of benzethonium chloride is in the range from 0.01% to 0.5% by weight, particularly preferably 0.02% to 0.3% by weight and very particularly preferably 0.04% to 0.2% by weight, calculated with respect to the total weight of the aqueous phase.

Another typical ingredient which is preferably present in the aqueous phase is glycerol. If glycerol is present, it is preferable when the total amount of glycerol is in the range from 0.1% to 8.0% by weight, particularly preferably 0.5% to 6.0% by weight and very particularly preferably 1.0% to 5.5% by weight, calculated on the basis of the total weight of the aqueous phase.

It is further preferable when the aqueous phase contains sodium chloride. Sodium chloride is preferably present in amounts of 0.1% to 0.8% by weight, particularly preferably 0.2% to 0.7% by weight and very particularly preferably 0.35% to 0.6% by weight, calculated on the basis of the total weight of the aqueous phase.

It has further been found that when the composition comprises sodium chloride in an amount in the range from 0.30% to 0.6% by weight and caprylyl/capryl glucoside and/or coco glucosides in an amount in the range from 0.13% to 0.19% by weight, calculated on the basis of the total weight of the aqueous phase, the formation of solid particles in the oil phase is reduced. Normally, such particles appear on the walls of the packaging.

According to the invention, the interfacial tension of the oil phase is determined with a K100 tensiometer from Krüss.

### The Parameters for the Measurement are Described Below:

Apparatus: K100 tensiometer (KRÜSS)
Correction: yes: density
Temperature: 20° C.

### Setup:

Measurement body: Krüss standard ring
Radius: 9,545 mm
Wire diameter: 0.37 mm

### Parameters:

Speed surface detection: 4 mm/min
Sensitivity surface—detection: 0.004 g
Speed search: 4 mm/min
Sensitivity search: 0.001 g
Speed measurement: 2 mm/min
Sensitivity search: 0.001 g
Immersion depth: 3.00 mm
Return travel: 10%
Values: 5—max. 20
Data collection: linear
   The Density Measurements of the Oil Phases were Conducted Under the Following Conditions:
Method: density (triplicate determination)
Apparatus: DMA 4500 density meter (Anton Paar)
Correction: yes: for viscosity<700 mPas
System: Oscillating U-tube (flexural resonator)
Temperature: 20° C.

In general, various oil compounds can be mixed in order to obtain the parameters described in claim 1. This can be seen through the exemplary formulations which comprise oil phases that are quite different, while these oil phases are optimized in order a specific interfacial tension and density as described above.

According to the invention, some oils may preferably be present in the formulations according to the invention.

In one aspect, the preferred oils are selected from the group consisting of isododecane, C15-19 alkane, isopropyl palmitate, ethylhexyl cocoate, dicaprylyl ether and ethylhexyl stearate.

Oils preferred in accordance with the invention are also ester oils, in particular ethylhexyl stearate, ethylhexyl cocoate, isodecyl neopentanoate, caprylic/capric triglyceride, cocoglycerides, isopropyl palmitate, dibutyl adipate, isoamyl laurate, isoamyl cocoate, propylene glycol dicaprylate, coco-dicaprylate/caprate, dicaprylyl carbonate, propylheptyl caprylate, decyl oleate, C12-15 alkyl benzoate and/or decyl cocoate.

It is further preferable when natural oils are present. Preferred natural oils are coconut oil, (sweet) almond oil, walnut oil, peach kernel oil, apricot kernel oil, avocado oil, tea tree oil, soybean oil, glycine soya oil, sesame oil, sunflower oil, tsubaki oil, evening primrose oil, rice bran oil, palm kernel oil, mango kernel oil, cuckooflower oil, safflower oil, macadamia nut oil, grape seed oil, amaranth oil, argan oil, bamboo oil, olive oil, wheat germ oil, pumpkin seed oil, mallow oil, hazelnut oil, safflower oil, rapeseed oil, sasanqua oil, jojoba oil, rambutan oil, cocoa butter, vegetable oil and shea butter. Preference is given to glycine soya oil and vegetable oil.

Further preferred oils are selected from ether oils, especially dicaprylyl ether.

It is further preferable when the amount of oils that are liquid under standard conditions is at least 80% by weight, more preferably 85% by weight, more preferably 90% by weight, more preferably 95% by weight, more preferably 98% by weight and most preferably at least 99% by weight, calculated on the basis of the total weight of the oil phase.

The oil phase and/or the aqueous phase may preferably comprise a dye or a color pigment in order to provide an attractive visual appearance.

### EXAMPLES

The following examples are intended to illustrate the compositions of this invention without limiting the invention to these examples. The numerical values in the examples are weight percentages, based on the total weight of each phase of the preparations.

The test results are reported in the following table with exemplary formulations. All examples with Coin. X, where X is an integer, are non-inventive reference examples. All formulations labelled with Ex.Y, where Y is an integer, are inventive examples.

| | INCI | Ex. 1 | Ex. 2 | Ex. 3 | Com. 1 | Com. 2 |
|---|---|---|---|---|---|---|
| Water phase | Sodium chloride | 0.32 | 0.48 | 0.3 | 0.55 | 0.34 |
| | Water | to 100 | to 100 | to 100 | to 100 | to 100 |
| | Biotin | | 0.02 | 0.02 | | |
| | Glycerol | 3.89 | 3.89 | 3.53 | 6.14 | 3.8 |
| | Benzethonium | 0.07 | 0.07 | 0.07 | 0.09 | 0.09 |
| | chloride | | | | | |
| | Phenoxyethanol | 0.52 | 0.52 | 0.53 | 0.69 | 0.71 |
| | EDTA | 0.17 | 0.17 | 0.17 | 0.49 | 0.45 |
| | Sodium | | | | 0.13 | |
| | Cocoamphoacetate | | | | | |
| | Sodium Hydroxide | | | q.s. | | |
| | Coco Glucoside | | | 0.14 | | |
| | Caprylyl/Capryl | 0.20 | 0.179 | | | 0.15 |
| | Glucoside | | | | | |
| Oil phase | Isodecyl | | | | | |
| | Neopentanoate | | | | | |
| | Caprylic/Capric | | | | 29.5 | |
| | Triglyceride | | | | | |
| | Coco glycerides | | | | | |
| | Isododecane | to 100 | to 100 | to 100 | | |
| | Isopropyl Palmitate | 7.24 | 7.24 | 8.4 | | |
| | Glycine Soja Oil | | | | | |
| | Dibutyl Adipate | | | | | |
| | Vegetable Oil | | | | | |
| | Isoamyl Laurate | | | | | |
| | Helianthus Annuus | | 0.02 | 0.02 | | |
| | Seed Oil | | | | | |
| | CI 61565 | | 0.0005 | 0.0005 | | |
| | CI 60725 | | 0.0006 | 0.0006 | | |
| | Isoamyl Cocoate | | | | to 100 | |
| | Propylene Glycol | | | | | to 100 |
| | Dicaprylate/Dicaprate | | | | | |
| | Coco- | | | | | |
| | Caprylate/Caprate | | | | | |
| | Dicaprylyl Ether | 2.52 | 2.52 | 5.4 | | 4.1 |
| | Dicaprylyl Carbonate | | | | 8.5 | |
| | Propylheptyl | | | | | |
| | Caprylate + Tocopherol | | | | | |
| | Hydrogenated | | | | | 47 |
| | Polydecene | | | | | |
| | Decyl Oleate | | | | | |
| | Ethylhexyl Stearate | | | 6.4 | | |
| | Ethylhexyl Cocoate | 3.9 | 3.9 | | | |
| | C12-15 Alkyl | | | | | |
| | Benzoate | | | | | |
| | Decyl Cocoate | | | | | |
| | C15-19 Alkane | 9.14 | 9.14 | 9.8 | | |
| Mixing ratio (v/v) | Water phase | 55 | 50 | 50 | 55 | 55 |
| | Oil phase | 45 | 50 | 50 | 45 | 45 |
| Parameters | Density (oil phase) | 0.76 | 0.77 | 0.77 | 0.88 | 0.87 |
| | g/cm3 | | | | | |
| | Interfacial tension | 38 | 38 | 34 | 23.2 | 24.6 |
| | (mN/m) against | | | | | |
| | water | | | | | |
| Observation | Separation 24 hours | Discrete | Discrete | Discrete | No clear | Phase |
| | after mixing the | phase | phase | phase | phase | boundary |
| | phases | boundary | boundary | boundary | boundary | visible, |
| | | visible | visible | visible | visible | but not |
| | | | | | | discrete |
| | Oil phase | Completely | Completely | Completely | Cloudy | Cloudy |
| | transparency 24 | transparent | transparent | transparent | oil phase | oil phase |
| | hours after mixing | to the | to the | to the | | |
| | the phases | human eye | human eye | human eye | | |
| | Water phase | Completely | Completely | Completely | Cloudy | Completely |
| | transparency 24 | transparent | transparent | transparent | water | transparent |
| | hours after mixing | to the | to the | to the | phase | to the |
| | the phases | human eye | human eye | human eye | | human eye |

The examples were analyzed in the following way. After sample preparation, the two-phase products were thoroughly shaken for 10 seconds and then left to stand without moving. 24 hours after shaking, the appearance of the phases was visually examined.

As can be seen from the table above, excellent results were obtained with the formulations according to the invention.

1-17. (canceled)

18. A two-phase cosmetic cleansing composition, wherein the composition is
not an emulsion and comprises
(a) an aqueous phase, and
(b) an oil phase,
a volume ratio between the aqueous phase and the oil phase being from 3:1 to 1:3, and the oil phase
having an interfacial tension with water of from 30 mN/m to 39 mN/m,
having a density of from 0.76 g/cm³ to 0.81 g/cm³, and
being free of silicone oils and silicone compounds.

19. The composition of claim 18, wherein the volume ratio between the aqueous phase and the oil phase is from 2:1 to 1:2.

20. The composition of claim 18, wherein the volume ratio between the aqueous phase and the oil phase is from 1.5:1 to 1:1.5.

21. The composition of claim 18, wherein the volume ratio between the aqueous phase and the oil phase is from 1.3:1 to 1:1.3.

22. The composition of claim 18, wherein the aqueous phase contains at least 60% by weight of water, based on a total weight of the aqueous phase.

23. The composition of claim 18, wherein the aqueous phase contains less than 99% by weight of water, based on a total weight of the aqueous phase.

24. The composition of claim 18, wherein the aqueous phase comprises one or more surfactants.

25. The composition of claim 24, wherein the aqueous phase comprises from 0.01% to 2% by weight of the one or more surfactants, based on a total weight of the aqueous phase.

26. The composition of claim 24, wherein the aqueous phase comprises from 0.02% to 1% by weight of the one or more surfactants, based on a total weight of the aqueous phase.

27. The composition of claim 24, wherein the aqueous phase comprises from 0.025% to 0.5% by weight of the one or more surfactants, based on a total weight of the aqueous phase.

28. The composition of claim 18, wherein the aqueous phase comprises one or more nonionic surfactants.

29. The composition of claim 28, wherein the aqueous phase comprises at least one alkyl glucoside as nonionic surfactant.

30. The composition of claim 29, wherein the aqueous phase comprises one or more of coco glucoside, caprylyl/capryl glucoside, decyl glucoside, and lauryl glucoside.

31. The composition of claim 28, wherein the aqueous phase comprises from from 0.02% to 1% by weight of the one or more nonionic surfactants, based on a total weight of the aqueous phase.

32. The composition of claim 28, wherein the aqueous phase comprises from from 0.1% to 0.5% by weight of the one or more nonionic surfactants, based on a total weight of the aqueous phase.

33. The composition of claim 18, wherein the aqueous phase comprises sodium chloride.

34. The composition of claim 33, wherein sodium chloride is present in a concentration of from 0.1% to 0.8% by weight, based on a total weight of the aqueous phase.

35. The composition of claim 18, wherein the composition comprises from 0.30% to 0.6% by weight of sodium chloride and from 0.13% to 0.19% by weight of caprylyl/capryl glucoside and/or coco glucoside, based on a total weight of the aqueous phase.

36. The composition of claim 18, wherein the composition comprises at least one oil selected from one or more of isododecane, C15-19 alkane, isopropyl palmitate, ethylhexyl cocoate, dicaprylyl ether, and ethylhexyl stearate.

37. The composition of claim 18, wherein a concentration of oils that are liquid under standard conditions is at least 80% by weight, based on a total weight of the oil phase.
